(19) United States
(12) Patent Application Publication
(10) Pub. No.: US 2010/0175692 A1
(43) Pub. Date: Jul. 15, 2010

(54) FORWARD METERING VALVE

(75) Inventors: Joergen Rasmussen, Struer (DK); Soeren Christrup, Struer (DK)

Correspondence Address: ROBERTS MLOTKOWSKI SAFRAN & COLE, P.C.;Intellectual Property Department, P.O. Box 10064, MCLEAN, VA, 22102-8064, US

(73) Assignee: BANG & OLUFSEN MEDICOM A/S, Struer (DK)

(21) Appl. No.: 11/914,080

(22) PCT Filed: May 8, 2006
(86) PCT No.: PCT/DK06/00245

§ 371 Date: Mar. 9, 2009

(30) Foreign Application Priority Data
May 10, 2005 (DK) PA 2005-00677
Feb. 3, 2006 (DK) PA 2006 00155

(51) Int. Cl. A61M 11/00

(52) U.S. Cl. 128/200.18

(57) ABSTRACT

A dose metering inhaler for administration via a liquid phase uses a rotating metering element that transports the metered dose from the pressurized canister to the mouthpiece. The metering is improved in that there is an efficient protection against penetration of outside air, and in that the counting of the doses is improved by preventing backwards rotation of the rotating metering element.

### BACKGROUND OF THE INVENTION

1. Field of the Invention

The present invention is directed to the types of inhalation devices widely used with medical drugs for the treatment of asthma, COPD, diabetes, systemic pain etc., which can be treated by inhalation of a drug.

2. Description of Related Art

Normally, two different types of medical drug formulations are used−one in which the drug is provided in dry powder form (dry powder inhaler=DPI), and one where the drug is mixed into a suitable propellant in pressurized, liquid form (pressurized metered dose inhaler=pMDI).

New regulatory issues require that both DPI's and pMDFs are equipped with a reliable dose indicator, indicating the number of doses left to the patient in the inhaler.

New developments of pharmaceutical formulations, where medical drugs are targeted to be administered via the lungs, especially in diabetes and pain relief, raise new requirements relative to the accuracy of dose metering and dynamic dose titration, such as rapid multiple sequential release of doses within the same inhalation period.

The metering valve should preferably involve few parts and be well suited for automatic assembly and low manufacturing costs.

In the pMDI, the medical drug is mixed into a propellant liquid and contained under pressure in a canister. To meter and release the drug in uniform doses, the canister is mounted with a metering valve, i.e., as disclosed in U.S. Pat. No. 3,756,465 to Meshberg. The common valve is a compress-and-release type of valve. From this, the more popular name “press-and-breathe” has been given to the pMDI (FIG. 1). While the patient inhales through the mouthpiece of the pMDI (11), the patient is supposed to manually compress and release the pMDI canister (12) to obtain the drug to be inhaled, illustrated by the curve in FIG. 2. Initial position (21) is to the left on the curve, where the canister is in a fully extended state.

While inhaling through the mouthpiece, the canister is manually compressed (22), passing the point of release of the previously metered dose (23) until it reaches its fully compressed state (24). After a certain delay (25), the canister is released (26), passing the point of metering of the next dose (27) until it again reaches the fully extended state (28) (reset).

A number of problems are known in present pMDI devices that may result in improper dose release:
A. During inhalation, the patient must be capable of overcoming the canister compression force of 30-50 N to release the previously metered dose of drug (23) (canister pressure typically 0.3-0.6 MPa). The majority of this force is required to compress a built-in return spring, strong enough to ensure the reset of the valve (28).
B. The patient must hold the pMDI upright during metering of the next dose (27)—after inhalation—otherwise a full dose of drug will not obtained at next use. The rationale for this is difficult to understand for the patient.
C. The delay (25) must not exceed 5-10 seconds, otherwise the metering of the next dose (27) will be inaccurate and the patient will not obtain a precise dose of drug at next use.

The main reasons for inaccurate dose releases are:
1. Metering from a non-uniformly dissolved solution. The user is instructed to shake the inhaler prior to inhalation in order to dissolve the drug uniformly in the liquid solution inside the canister. If too long time elapses from shaking to metering of the next dose, the solution becomes non-uniformly dissolved, resulting in a too low or too high dose of drug metered for next use.
2. Atmospheric air trapped inside the metering chamber. By delaying the metering of next dose after delivery of the present dose, air will migrate from the outside into the metering valve. The air will be trapped inside the metering valve taking up volume, resulting in the metered dose being too small.

Furthermore, there is a risk of drug leaking from the canister to the outside during the delay, as a smaller load is applied to the gaskets inside the metering valve during canister compression. This can lead to a serious lack of drug, when needed by the user.

To overcome the problem of (A) and to improve the coordination between inhalation and dose delivery, breath actuated inhalers (BAFs) have been developed. However, a reset must be performed manually by the patient after inhalation, e.g., by closing the cap of the BAI, so that the risks of (B) and (C) get seriously worse.
D. If the time between doses is too long (e.g. some days) there is a risk of degradation of the metered dose (loss of prime). It is required that the patient performs priming shots, both before first use and before use if the pMDI has not been used for some days or longer.
E. Adding a reliable dose indicator to a pMDI or BAI introduces new problems. As the magnitude of the position tolerance of the canister is comparable to the canister stroke during compression and release, it is not obvious how to design a tamper-proof dose indicator which never counts too many doses AND never counts too few doses.

Canister Filling Issues:
F. Generally, canister mounted metering valve designs must comply with the industry practice of factory filling of the canister with drug and propellant. It is common procedure that the metering valve is mounted on the canister before filling. Therefore, the valve design must allow for reverse flow, when high pressure is applied to the metering valve outlet to fill the canister.

Rotational metering valves are well known from prior art relating to dry powder inhalers such as UK Patent Application GB 2165159 to Auvinen. However, these valves are pressure and sealing free, and depend on gravity only.

Rotational dose metering devices for fluids are known from, i.e., gasoline pumps, and within the medical field some examples has been disclosed in U.S. Pat. No. 6,179,583 to Weston and U.S. Pat. No. 6,516,796 to Cox. These valves are designed to work with propellant-free liquids at low pressures, they are complicated and expensive to manufacture and have not been demonstrated to work at the typical canister pressure of O.3-0.6 MPa.

### SUMMARY OF THE INVENTION

This invention relates to a sealed unidirectional rotational metering valve with fixed metering cavities to be used with pressurized canister based aerosol inhalers. The invention solves several of the above mentioned problems inherent with existing pressurized aerosol inhalers:

Activation force is minimized as the metering valve does not need a preloaded return spring.

The metering valve is filled and the metered dose is released in one actuation movement after the user has placed the inhaler in upright position for oral or nasal application. Therefore, a full dose will be reliably and accurately metered and problems with long term migration of a former metered dose are avoided.

The need for reset time is obviated, as the liquid in the canister will flow freely into the metering cavity.

As the unidirectional rotational motion of the metering valve is unambiguously well suited for a simple counter mechanism, a reliable visual dose indicator is easily attached to the valve.

An embodiment of an elastic valve sealing member is disclosed that allows for standard canister filling procedures.

The forward metering valve can be part of the drug canister or it can be an add-on device to the drug canister.

A further aspect of the invention is that the forward metering valve is extremely suitable for multi-dose operation, because the mechanical movement of the valve can be rotational, continuous and unidirectional.

### BRIEF DESCRIPTION OF THE DRAWINGS

FIG. 1 shows a conventional pMDI inhaler.

FIG. 2 shows the timing associated with a conventional inhaler.

FIG. 3 shows an embodiment of the forward metering valve in sectional and perspective views.

FIG. 4 shows the functional steps of the forward metering valve involved during one inhalation action.

FIG. 5 shows the positioning of an optional one-way valve in open and closed positions.

FIG. 6 is a perspective view of an embodiment of the attachment of a dose counter wheel.

FIG. 7 is a perspective view of an embodiment of a backward locking mechanism.

FIG. 8 is a perspective view of an embodiment of a step locking mechanism.

FIG. 9 is a perspective view of an embodiment of a gear wheel actuation mechanism.

FIG. 10 is a perspective view of an embodiment of a pushbutton/ratchet mechanism.

FIG. 11 is a perspective view of an embodiment of a breath actuated dose release mechanism.

FIG. 12a is a partial sectional view of an embodiment of the forward metering valve integral within a canister

FIG. 12b is a partial sectional view showing one principle of filling the canister through the forward metering valve

FIG. 13 is a diagrammatic perspective view of an inhaler configuration, where a forward metering valve is driven by a battery powered motor under control of a breath activated mechanism and a control unit.

FIG. 14 is a graph showing the timing of a single dose release with a breath activated release mechanism.

FIG. 15 is a graph showing the timing of a multi-dose release with a breath activated release mechanism.

FIG. 16 is a graph showing the timing of an adaptive multi-dose release controlled by continuous measurement of inhalation flow

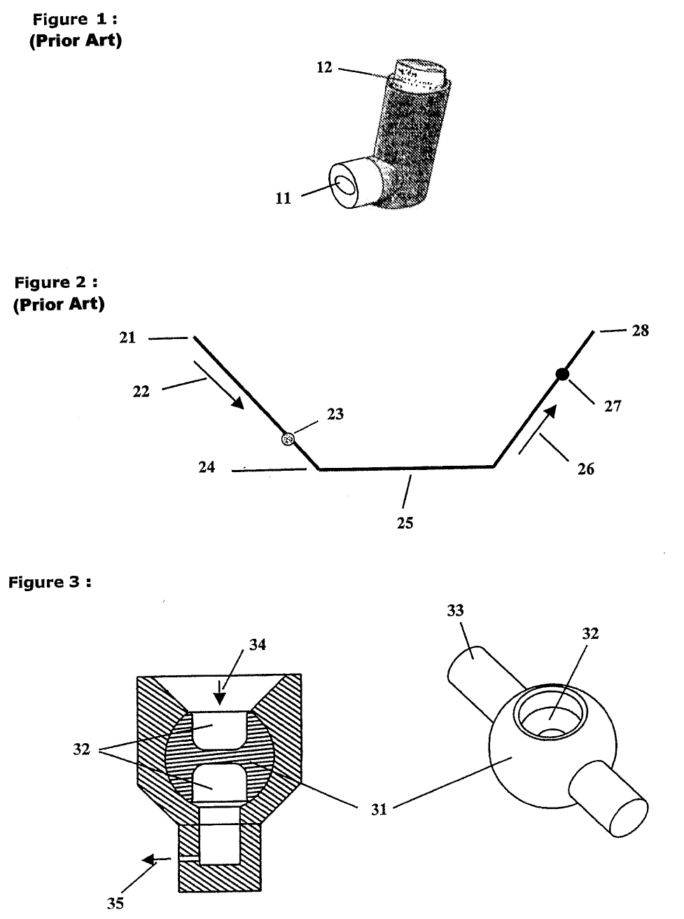

### DETAILED DESCRIPTION OF THE INVENTION

One possible embodiment of the forward metering valve of the present invention is shown in FIG. 3. The valve rotor (31) shown is ball shaped. Other shapes are possible, e.g., cylinder shaped, as long as the shape is rotationally symmetric. The valve rotor contains one or more metering chambers (32). During inhalation the rotor shaft (33) is turned, rotating one metering chamber from the inlet from a pressurized drug container (34) to the outlet through a nozzle (35).

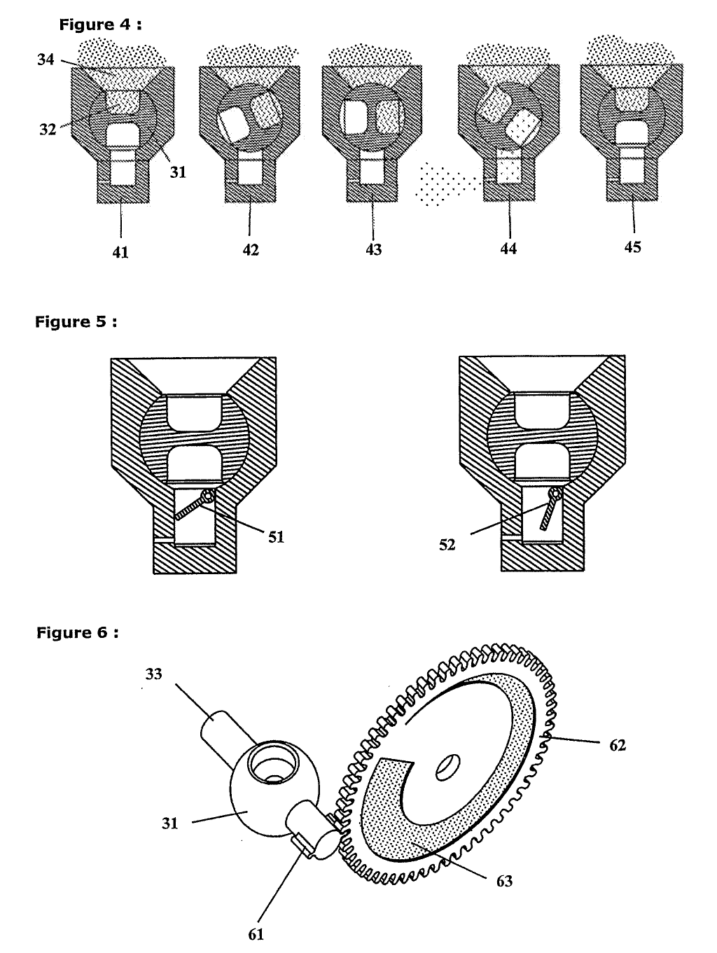

The valve cycles during inhalation are shown in FIG. 4.

From an initial position (41) the valve rotor (31) is rotated clockwise to the metering position (42) where the metering chamber (32) is isolated from the inlet (34). After passing the half-way position (43) where the metering chamber is fully closed to the surroundings, the dose release (44) happens when the metering chamber opens up towards the outlet. The last cycle is the stop position (45), which, at the same time, is the initial position for the next dose. The embodiment shown will rotate approximately 180° to release a dose (2 doses per 360° rotation). Other options are 1, 3, 4, 5, 6 and more doses per 360° rotation.

As the current dose is metered within seconds ahead of delivery, problems (C) and (D) are obviated. There will be no need for priming shots.

Due to the lack of a return spring, the force to actuate the metering valve will be significantly lower than 30-50 N, and the effects of problem (A) will be significantly reduced.

As metering of the current dose is performed during inhalation, it is required to keep the inhaler upright during inhalation. This is far easier to understand for the patient than keeping the inhaler upright after inhalation, decreasing the effect of problem (B).

A potential problem with the proposed valve design is the possibility of feeding outside air and impurities into the pressurized drug bulk, when rotating an emptied metering chamber forward to the inlet position. This can be solved by adding a one-way valve to the outlet of the metering valve, preventing outside air to enter the emptied metering chamber. One possible embodiment of an additional one-way valve is shown in FIG. 5. Normally, the one-way valve will be in its closed position (51), allowing no outside air to enter the metering chamber. During dose release, the one-way valve will open up (52), allowing the drug to escape from the metering chamber through the nozzle to the outside.

In the case of the forward metering valve being integral with the canister, the one-way valve may be placed in an attached nozzle member, still allowing for standard canister filling procedures.

Adding a dose indicator to the proposed valve design will be a simple task. Because the metering valve is only intended to move in one direction, the dose counter can be continuously engaged with the valve and synchronized with the valve movement, eliminating the position tolerance problem and the effects of tampering according to problem (E). One possible embodiment of a dose indicator is shown in FIG. 6. The rotor gear wheel (61) is engaged with the indicator gear wheel (62), ensuring a fixed relation between the number of valve rotations and the position of the visual dose indicator (63). The visual dose indicator (63) can provide visualization of the remaining drug level in the canister by a patterned or colored field as shown, or it can be fitted with numbers or codes to indicate the approximate or precise number of doses left in the canister.

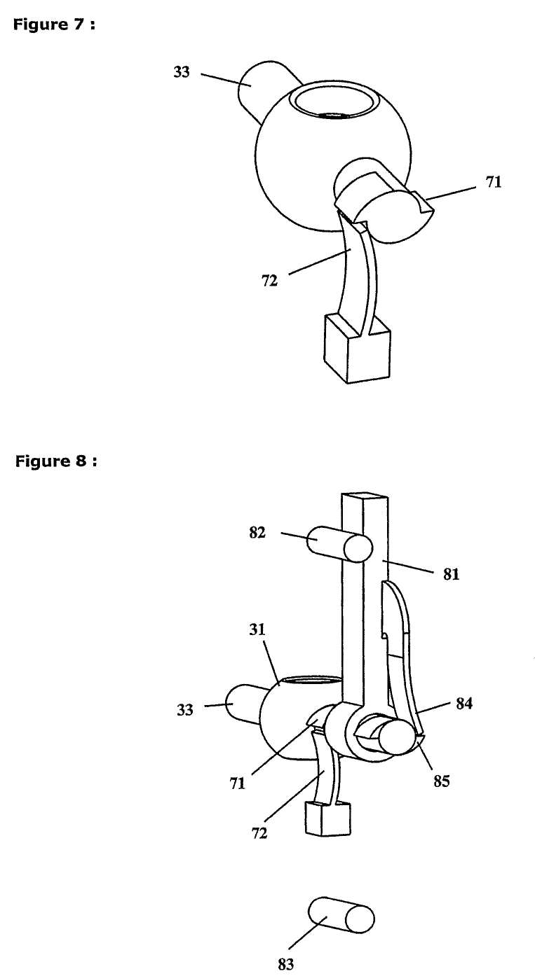

A potential risk of the proposed valve design in combination with the proposed dose indicator design is the risk of moving the valve backwards, releasing doses while turning the dose indicator backwards. This will lead to lack of synchronization between the dose indicator status and the actual amount of drug left in the container, which is a serious malfunction of a drug dose indicator. One possible embodiment of a backwards rotation lock is shown in FIG. 7.

When adding a backwards lock ratchet (71) to the rotor shaft (33) and a backwards lock spring (72) to the inhaler chassis it will become impossible to move the valve backwards, eliminating the risk of undercounting.

Another potential risk with the proposed valve design is the risk of releasing more doses than required per inhalation. To prevent this, a step lock can be applied. It will ensure that the valve will stop rotating after the required number of doses has been released during inhalation. The step lock can be realized in different embodiments. One possible option is shown in FIG. 8, releasing one dose per actuation.

The valve actuator (81) is mounted free-rotating on the rotor shaft (33). To actuate the valve and release one dose, the valve actuator must be moved clockwise from its upright position resting against the actuator reverse stop (82) to its downwards position stopped by the actuator forward stop (83). During this, the step lock spring (84) will engage the step lock ratchet (85), rotating the rotor shaft (33) and the valve rotor (31) forward.

To prepare the valve for the next dose, the valve actuator (81) must be returned to its upright position, resting against the actuator reverse stop (82). During this, the backwards lock spring (72) will engage the backwards lock ratchet (71), ensuring that the valve rotor (31) will not rotate backwards.

Delivering a single dose with the rotational valve requires a rotational input to the valve shaft to actuate the valve during inhalation.

Basically, the valve rotation can be actuated in two different ways:
1. Manual actuation
2. Breath actuation

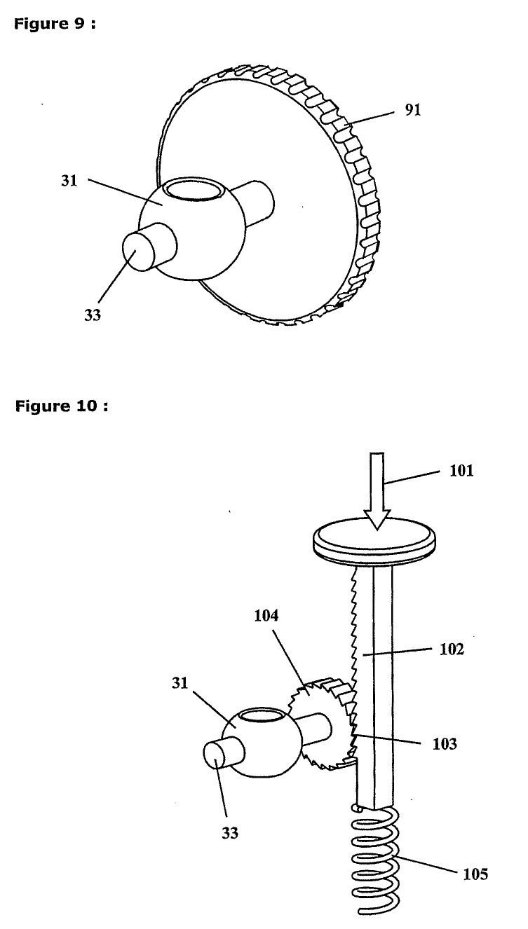

Manual actuation can be obtained by requiring the user to manually actuate the valve rotation. One possible embodiment is shown in FIG. 9, where a finger wheel (91) is mounted directly onto the rotor shaft (33) to directly rotate the valve rotor (31) and thereby releasing a dose of drug.

Another possible embodiment is shown in FIG. 10, where a mechanism requires the user to perform a linear input movement (101) to rotate the valve rotor (31). During user input, a rack (102) travels down. The pinion (103) is engaged with a rotor gear wheel (104) mounted on the rotor shaft (33), causing the valve rotor (31) to rotate clockwise and thereby releasing a dose of drug. After inhalation, a return spring (105) can cause the rack (102) to return to the initial position without causing the valve rotor (31) to rotate counter-clockwise, by performing as a ratchet.

Breath actuation can be obtained by using stored energy to actuate the valve rotation. The stored energy is triggered by the user's inhalation through the inhaler. The energy can be stored in several ways.

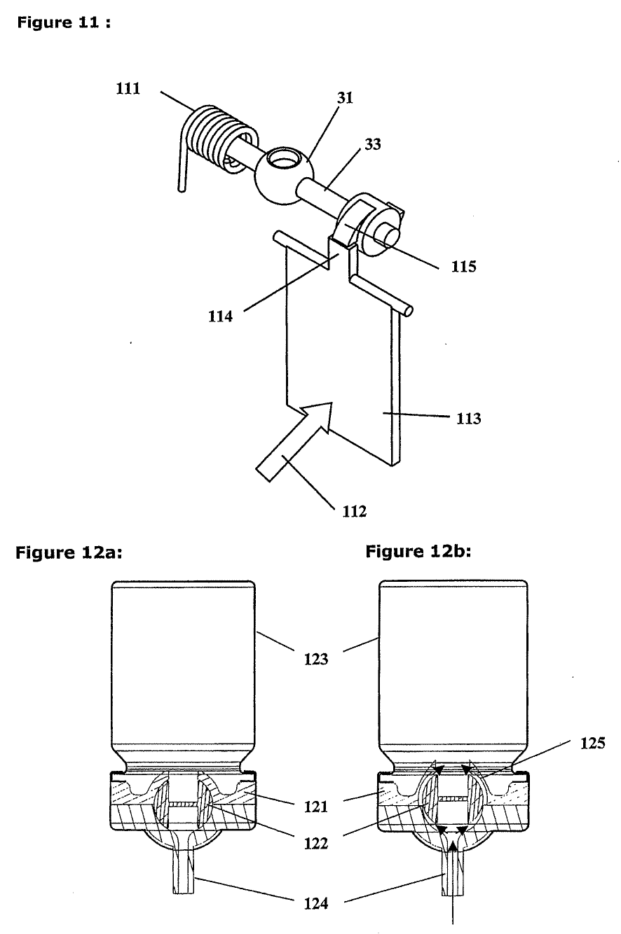

In FIG. 11 one possible embodiment is shown, where energy stored in a loaded spring (111) is applied to the rotor shaft (33), directly rotating the valve rotor (31) counter-clockwise and thereby releasing a dose of drug. To trigger the loaded spring (111) by the user's inhalation flow (112), a hinged flap (113) is mounted in the airflow path of the inhaler, causing the flap lock (114) to release the rotor lock (115) so that the loaded spring (111) is allowed to rotate the valve rotor (31) and thereby release a dose of drug.

FIG. 12 shows one embodiment of a ball shaped forward metering valve (122) being integral with a canister (123). In this embodiment, with two metering cavities, an inhalation procedure results in a 180° rotation of the valve. In the case of the forward metering valve being integral with the canister, the valve must allow for reverse flow through the valve, whenever a pressurized liquid is applied to the valve outlet (124). One embodiment is shown on FIG. 12a, where an elastic sealing member (121) effectively seals the pressurized liquids in the canister (123) from leaking through the valve. In the canister filling situation of FIG. 12b, the pressure at the valve outlet (124) is higher than the pressure inside the canister. This pressure difference will force the sealing member to open (125) in the same way as an inflation valve in a bicycle inner tube and therefore allow the pressurized liquid to flow into the canister until an equilibrium pressure is present across the valve. Problem (F) is therefore solved.

A further aspect of the invention is that the forward metering valve disclosed here is extremely suitable for multi-dose operation, because the mechanical movement of the valve is rotational and unidirectional. The metering cavities can be filled and emptied during rotation at reasonable turning speeds, thus allowing several metered doses to be released during an inhalation sequence.

This aspect reveals several applications that could solve some problems related to administration of drugs:

Pulmonary administration of insulin is a promising new drug delivery therapy. Unlike most asthma inhalers that deliver the same dose every time, insulin inhalers must be able to preset and deliver different dose sizes dependent of time of day, meals intake, and exercise levels.

Pulmonary administration of pain killers for patients having chronic pain also requires adjustment of doses to the actual pain level.

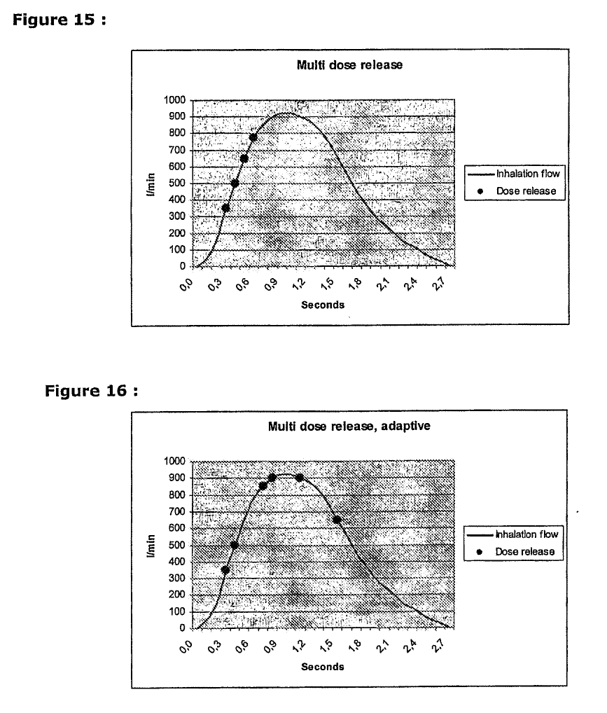

Inhaler research has indicated that it is advantageous for optimal drug deposition to release smaller dose portions during the inhalation sequence shown in FIG. 15 instead of one big puff in the beginning of the inhalation sequence shown in FIG. 14. This can easily be achieved by the forward metering valve by choosing the right relation between metering cavity volume, valve rotation speed and number of released doses.

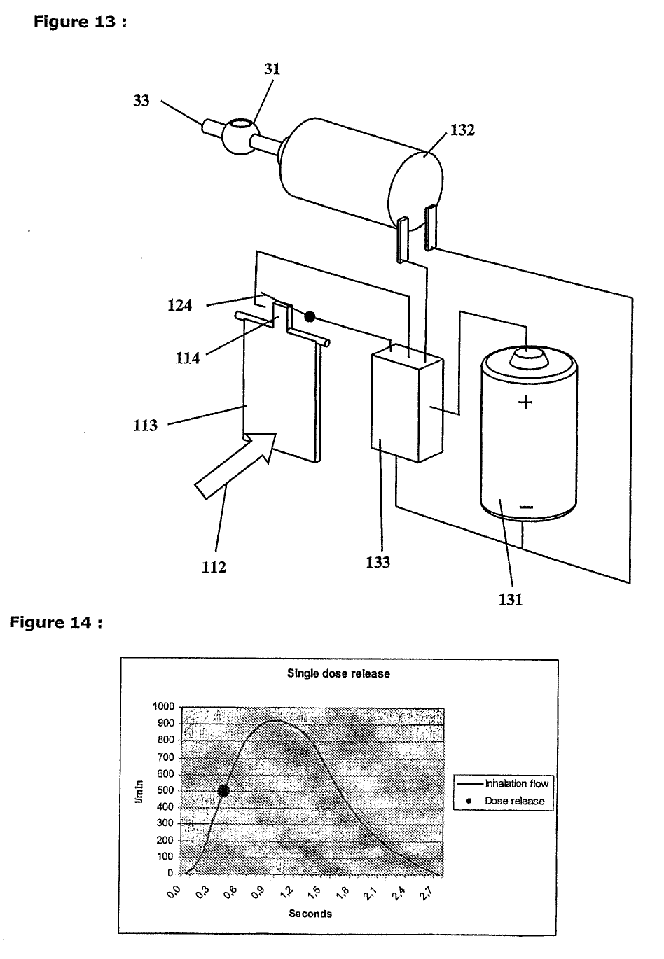

A further aspect of the invention is that the unidirectional rotation of the forward metering valve is easily connected to and driven by a simple motor as shown in FIG. 13. The battery (131) powered motor (132) may be controlled by timing alone or more advantageously by measuring the actual inhaler flow in the flow channel and adapt the dose release pattern to the actual inhalation flow profile FIG. 16. The flow sensor could be, e.g., of the differential pressure type, hot wire anemometry type or even a mechanical displacement type sensor (113). Besides valve release control, the controller (133) might also solve safety issues like reliable dose counting and overdose protection by disabling dose releases in a certain period of time after a successful inhalation sequence has been performed.

1-15. (canceled)

16. A metering valve for an inhalation device, comprising:
a valve inlet and a valve outlet,
a rotor shaft connected to actuating means for rotating the rotor shaft,
a valve rotor comprising at least one metering cavity, being a part of said rotor shaft, and being rotatable in only one direction, and
a dose indicator mechanism directly connected to said rotor shaft
wherein said at least one metering valve cavity is adapted to be filled with a dose and to subsequently release said dose during the same actuation of the metering valve.

17. A metering valve according to claim 16, wherein said metering valve inlet has a sealed connection to a canister, said canister containing a medium under pressure, and in which said valve rotor is rotationally connected between the canister and the outlet such that the metering chamber may be rotated from a position in communication with the medium inside the canister to a position, in which said metering chamber is in communication with the outlet.

18. A metering valve according to claim 17, wherein the valve rotor is built into the canister.

19. A metering valve according to claim 18, wherein said valve rotor comprises a rigid valve rotor element and wherein said valve further comprises and an elastic gasket member sealingly mounted in association with the rigid valve rotor element, wherein the valve has the following three modes of operation:
a first mode in which a linear movement of the valve rotor element in the gasket member permits entry of fluid into the canister from the outlet when said fluid is under greater pressure than the inside of the canister,
a second mode in which valve rotor element is stationary sealing the canister content from the outside by means of the gasket member when the pressure inside the canister is greater than the surroundings, and
the third mode in which the rotor is rotated in said gasket, performing a metering action and delivering a metered dose from the inlet to the outlet.

20. A metering valve according to claim 19, wherein, in the first mode of operation, the gasket member is deflected by the fluid and the valve rotor in such a manner that the entry of fluid is facilitated.

21. A metering valve according to claim 16, wherein said rotating valve is rotationally symmetrically shaped.

22. A metering valve according to claim 16, wherein a one-way valve is arranged between the valve rotor and the outlet, the one-way valve preventing a medium from flowing into the metering chamber through the outlet.

23. A metering valve according to claim 17, wherein the dose indicator visually displays the approximate number of doses left in the said canister and is connected to the rotor shaft by a gear mechanism.

24. A metering valve according to claim 16, wherein the valve rotor is connected to a locking means having a locking spring and a backwards locking ratchet, and wherein said locking spring interacts with the backwards locking ratchet to ensure said one-way rotation of the valve rotor.

25. A metering valve according to claim 16, wherein the valve rotor is connected to a mechanical transmission means, said mechanical transmission means being connected to an activation means for generating rotational movement of the valve rotor upon activation of said activation means.

26. A metering valve according to claim 16, wherein the metering valve comprises an activation means, and wherein said activation means is connected to an actuating means.

27. A metering valve according to claim 26, wherein said activation means comprises a breath actuated member, said breath actuated member being connected to a trigger means for controlling said actuating means.

28. A metering valve according to claim 16, further comprising control means for controlling the number of doses or sub-doses metered for each inhalation by a user, said control means stopping the valve rotor from rotating after a defined number of doses or sub-doses have been metered.

29. A procedure for operating a metering valve having a valve inlet and a valve outlet, a rotor shaft connected to actuating means for rotating the rotor shaft, a valve rotor that has at least one metering cavity, is a part of said rotor shaft, and is rotatable in only one direction, and a dose indicator mechanism directly connected to said rotor shaft, wherein the at least one metering valve cavity is adapted to be filled with a dose and to subsequently release the dose during the same actuation of the metering valve, comprising the steps of:
initially, connecting the metering cavity of the valve rotor to a volume containing a medium under pressure,
second, rotating the valve rotor to close the metering cavity, and
third, rotating the valve rotor further to connect the metering cavity to the outlet

30. A procedure for operating a forward metering valve according to claim 29, comprising the further step of:
initiating of an activation step by user inhalation and the valve rotor being rotated a given number of turns causing at least one metering chamber to be emptied a given number of times to deliver a predetermined dose of medicine to the user.

31. A procedure for operating a forward metering valve according to claim 29, comprising the further step of:
providing a canister containing a medium under pressure to which said metering valve is in a sealed connection to a canister,
producing rotation between the canister and the metering cavity from a position in which the metering cavity is in communication with the medium inside the canister to a position in which said metering cavity is in communication with the outlet, and
indexing the dose indicator mechanism by rotation of the rotor shaft to indicate the number of doses that remain in the canister.
